(19) United States
(12) Patent Application Publication
(10) Pub. No.: US 2025/0065081 A1
(43) Pub. Date: Feb. 27, 2025

(54) ARTICULATING CATHETER SYSTEM AND METHOD

(71) Applicant: NP Medical Inc., Clinton, MA (US)

(72) Inventors: Todd Chelak, Pelham, NH (US); Nicholas Illsley, Sterling, MA (US); Ian Kimball, Lunenburg, MA (US); John Damarati, Marlborough, MA (US); Andrew Galanis, Ipswich, MA (US)

(21) Appl. No.: 18/724,500

(22) PCT Filed: Dec. 28, 2021
(86) PCT No.: PCT/US2021/065278

§ 371 Date: Jun. 26, 2024

(51) Int. Cl. A61M 25/00; A61M 39/10

(52) U.S. Cl. CPC A61M 25/0097; A61M 25/0043; A61M 39/105; A61M 2025/0059; A61M 2039/1033; A61M 2205/0216; A61M 2210/12

(57) ABSTRACT

A catheter system includes a catheter hub, a catheter shaft and a hub housing. The catheter hub has a flow path extending from an opening at a distal end to an inlet at a proximal end. The catheter shaft has a lumen in fluid communication with the distal end opening of the catheter hub. The hub housing has a fluid path extending through a portion of it. A cavity located distal to the fluid path has a wall with an opening that movably retains the catheter hub. The surface of the catheter hub and the wall of the cavity movably interact from a first position to at least a second position. The longitudinal axis of the fluid path distal opening is parallel with the longitudinal axis of the lumen when in the first position and forms an angle with the longitudinal axis of the lumen when in the second position.

### TECHNICAL FIELD

The present invention relates to catheter systems, and more particularly to articulating catheter systems that help prevent catheter shafts from becoming kinked when placed within the patient's vasculature.

### BACKGROUND ART

In instances in which a patient will need regular administration of fluid or medications (or regular withdrawal of fluids/blood), catheters are often inserted into the patient and used to administer the fluids/medications. The catheter may remain in the patient for extended periods of time (several hours to several days or longer). Additionally, an extension tube may be connected to the catheter to facilitate use of the catheter and connection of a medical implement (e.g., a syringe). Alternatively, the medical implement may be connected directly to a luer connector (or similar connector) at the end of the catheter (see FIGS. 1A to 1E). To ensure that the catheter and/or extension tube remain in place and are not accidentally removed, some prior art systems attempt to secure the catheter and/or extension tube to the patient using tape or similar adhesive materials (e.g., a film dressing).

Once the catheter is in place in the patient's vasculature, prior art catheters may become kinked, for example, at a bend/kink point between the connector and the insertion site (see FIGS. 1B and 1E). Unintentional movement of the catheter in the direction of insertion may act on the bend/kink point to further collapse the catheter (see FIG. 1E). This kinking may compromise flow rates (delivery and withdrawal), lead to IV pump alarms and/or inability to withdraw blood. The kinking may occur often and tends to be more frequent with increased use of ultrasound guided placement (deeper veins/steeper angles), longer and/or softer catheter shafts, placement within the antecubital or AC (easy access, bend point), and/or placement within the hand/wrist (bend point).

### SUMMARY OF THE INVENTION

In accordance with one embodiment of the invention, a catheter system for conforming to a patient's body includes a catheter hub having a proximal end, a distal end, an exterior surface, and an internal flow path extending from an opening at the distal end to an inlet at the proximal end. The flow path allows fluid to flow between the distal end and the proximal end of the catheter hub. The system may also have a catheter shaft and a hub housing. The catheter shaft may have a lumen with a longitudinal axis, and the lumen may be in fluid communication with the opening at the distal end of the catheter hub. The hub housing has a bottom surface, an internal fluid path extending through at least a portion of the hub housing, and a cavity located distal to the fluid path. The cavity may have a wall with an opening, and the wall of the cavity may movably retain the catheter hub. The fluid path may have a distal opening with a longitudinal axis and may be fluidly connected to the inlet of the flow path in the catheter hub when the catheter hub is retained within the cavity of the hub housing. The opening at the distal end of the catheter hub may be located proximate the opening of the cavity wall. The external surface of the catheter hub and the wall of the cavity movably interact and maintain a fluid tight seal therebetween from a first position to at least a second position. The longitudinal axis of the fluid path distal opening may be parallel with the longitudinal axis of the catheter shaft lumen when in the first position. The longitudinal axis of the fluid path distal opening may form an angle with the longitudinal axis of the catheter shaft lumen when in the second position and when the bottom surface of the hub housing is placed upon the patient's body.

In some embodiments, the catheter system may also include a retaining member that extends at least partially through the hub housing. The retaining member transitions between a locked position and an unlocked position, and may selectively lock the catheter hub in the first or second position when in the locked position. The retaining member may act on the catheter hub when in the locked position to press the catheter hub against a retaining surface, thereby locking the catheter hub. For example, the retaining surface may be an internal surface within the hub housing.

In accordance with further embodiments, the hub housing may also have a proximal port located at a proximal end of the hub housing. The proximal port may include a seal member located within the proximal port to seal the proximal port. The proximal port may include a luer connector. An introducer may connect to the proximal port of the hub housing. The introducer has a needle extending through the internal fluid path, the internal flow path, and the lumen of the catheter shaft when the introducer is connected to the proximal port. The introducer may allow the catheter to be inserted into a patient's vasculature when connected and may be disconnected from the proximal port after insertion of the catheter into the patient's vasculature. The hub housing may include at least one retention element that extends from an exterior surface of the hub housing. The at least one retention element may engage the introducer to secure the introducer to the hub housing when connected.

The external surface of the catheter hub includes at least one interacting surface extending from the external surface. The at least one interacting surface contacts the wall of the cavity to maintain the fluid tight seal. For example, the at least one interacting surface may be a resilient material overmolded on the catheter hub. Additionally or alternatively, the exterior surface of the catheter hub may be aligned with the wall of the cavity to maintain the fluid tight seal.

In accordance with additional embodiments, the hub housing may include a side port that may be fluidly connected to an extension tubing system. The extension tubing system may include a needle free connector and a tube. The needle free connector may have an inlet, an outlet and a priming port. The tube may have a first end fluidly connected to the outlet of the needle free connector and a second end connected to the side port of the hub housing. In some embodiments, the opening may be shaped to receive the catheter hub such that the catheter hub may be connected and/or disconnected from the hub housing.

In accordance with further embodiments, a method for connecting a catheter to a patient's vasculature includes providing a catheter system that has a catheter hub, a catheter shaft, and a hub housing. The catheter hub may have a proximal end, a distal end, an exterior surface, and an internal flow path extending from an opening at the distal end to an inlet at the proximal end. The flow path may allow fluid to flow between the distal end and the proximal end of the catheter hub. The catheter shaft has a lumen with a longitudinal axis, and the lumen may be in fluid communication with the opening at the distal end of the catheter hub. The hub housing has a bottom surface, an internal fluid path extending through at least a portion of the hub housing, and a cavity located distal to the fluid path. The cavity has a wall with an opening, and the wall may movably retain the catheter hub. The fluid path has a distal opening with a longitudinal axis and may be fluidly connected to the inlet of the flow path in the catheter hub when the catheter hub is retained within the cavity of the hub housing. The opening at the distal end of the catheter hub may be located proximate the opening of the cavity wall. The method may also include inserting the catheter shaft into the patient's vasculature, and rotating the catheter hub with respect to the hub housing from a first position to a second position. The longitudinal axis of the fluid path distal opening is parallel with the longitudinal axis of the catheter shaft lumen when in the first position. The longitudinal axis of the fluid path distal opening forms an angle with the longitudinal axis of the catheter shaft lumen when in the second position and when the bottom surface of the hub housing is placed upon the patient's body.

In some embodiments, the external surface of the catheter hub and the wall of the cavity may movably interact and maintain a fluid tight seal therebetween during rotation from the first position to the at least a second position. The hub housing may include a proximal port located at a proximal end of the hub housing and/or inserting the catheter shaft into the patient's vasculature may include using an introducer to insert the catheter shaft into the patient's vasculature. The introducer may be connected to the proximal port of the hub housing and may have a needle extending through the internal fluid path, the internal flow path, and the lumen of the catheter shaft when the introducer is connected to the proximal port. The method may then insert the needle and catheter shaft into the patient's vasculature, and disconnect the introducer from the proximal port of the hub housing after the catheter is inserted into the patient's vasculature. Additionally or alternatively, the method may transition a retaining member from an unlocked position to a locked position. The retaining member may extend at least partially through the hub housing and lock the catheter hub in the second position when in the locked position.

### BRIEF DESCRIPTION OF THE DRAWINGS

The foregoing features of embodiments will be more readily understood by reference to the following detailed description, taken with reference to the accompanying drawings, in which:

FIGS. 1A to 1E schematically shows a representative prior art catheter inserted into a patient.

FIG. 2A schematically shows a perspective view of a catheter system in accordance with various embodiments of the present invention.

FIG. 2B schematically shows a top view of the catheter system shown in FIG. 2A, in accordance with some embodiments of the present invention.

FIG. 2C schematically shows a cross-sectional view of the catheter system shown in FIG. 2A, in accordance with various embodiments of the present invention.

FIGS. 3A to 3C schematically show a side view and cross-sectional views of the catheter system shown in FIG. 2A with the introducer removed, in accordance with some embodiments of the present invention.

FIGS. 3D and 3E schematically show cross-sectional views of the catheter system shown in FIG. 2A with the introducer removed and with the catheter in different positions, in accordance with various embodiments of the present invention.

FIG. 3F schematically shows the catheter hub of the catheter system shown in FIG. 2A, in accordance with some embodiments of the present invention.

FIG. 3G schematically shows a cross-sectional view of the catheter system shown in FIG. 2A with the retaining member in the locked position, in accordance with some embodiments of the present invention.

FIG. 4 schematically shows a cross-sectional view of an alternative catheter system, in accordance with some embodiments of the present invention.

FIG. 5 schematically shows a perspective view of an additional alternative catheter system, in accordance with further embodiments of the present invention.

FIGS. 6A to 6C schematically show perspective views and a top view of an additional alternative catheter system, in accordance with further embodiments of the present invention.

FIGS. 6D and 6E schematically show cross-sectional views of the catheter system shown in FIGS. 6A to 6C, in accordance with additional embodiments of the present invention.

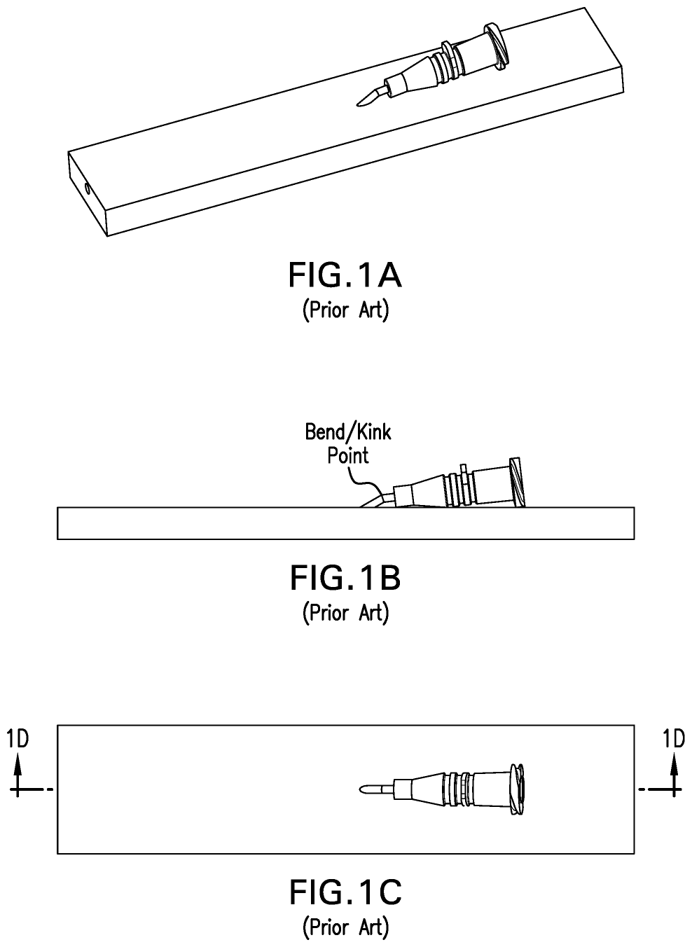

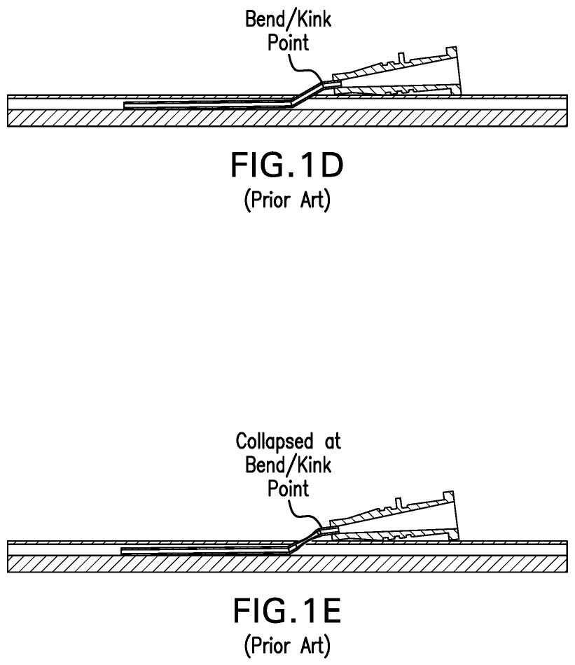

### DETAILED DESCRIPTION OF SPECIFIC EMBODIMENTS

In illustrative embodiments, a catheter system has a catheter hub with an internal flow path that extends from an opening at the distal end of the hub to an inlet at the proximal end. A hub housing has a cavity with a wall having an opening. The wall of the cavity movably retains the catheter hub. The catheter hub is movable from a first position to a second position to reduce stress on a catheter shaft attached to the catheter hub and reduce the risk of kinking of the catheter. Illustrative embodiments of the present invention are discussed in greater detail below.

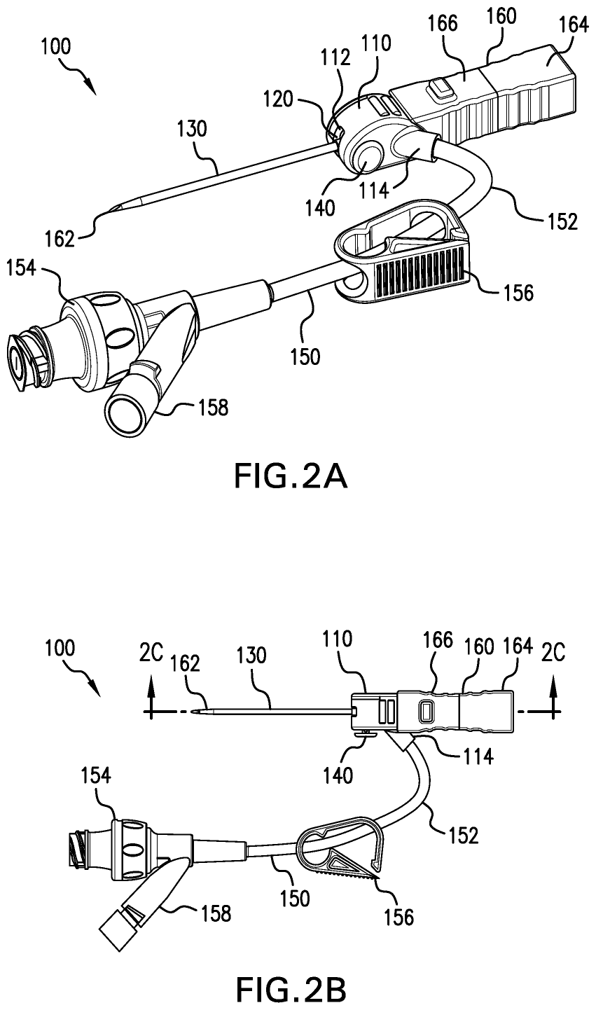

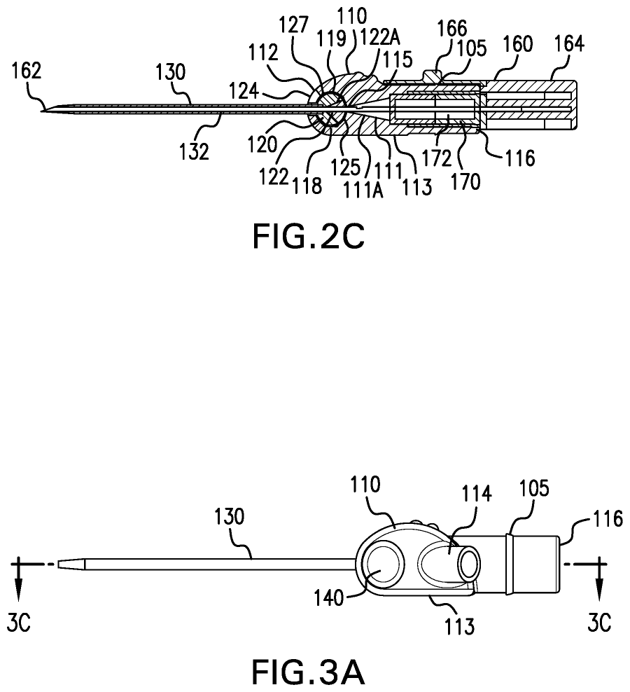

FIGS. 2A, 2B and 2C schematically show a perspective view, a top view and a cross-sectional view of a catheter system 100 in accordance with some embodiments of the present invention. The catheter system 100 includes a hub housing 110 that houses a catheter hub 120 that, in turn, has a catheter 130 extending from it. The hub housing 110 has an opening 112 that allows the catheter 130 to extend out of the hub housing 110 so that it may be inserted into the patient's vasculature. As discussed in greater detail below, the catheter hub 120 may rotate/articulate within the hub housing 110. As also discussed in greater detail below, the system 100 may have a retaining member 140 that locks the catheter hub 120 in position. To facilitate fluid flow through the catheter system 100, the catheter hub 120 may include an internal flow path 122 and the hub housing 110 may include an internal fluid path 111 extending at least partially though the hub housing 110.

In some embodiments, the hub housing 110 may include a side port 114 extending from one side of the housing 110. The side port 114 may connect to an extension tubing system 150 that, in turn, may be used to introduce fluid into and/or withdraw fluid from the patient via the catheter system 100. For example, the extension tubing system 150 may include a tube 152 that is connected to the side port 114 at one end and a needle free connector 154 at the other end. To selectively allow and prevent fluid through the tube 152, the extension tubing system 150 may also include a valve 156 (e.g., a clamp valve) located on the tube 152. When the valve 156 is open, fluid is allowed to flow through the tube 152. When the valve 156 is closed, fluid is not allowed to flow through the tube 152. To withdraw fluid from the patient and/or introduce fluid into the patient, a user may connect a medical implement (e.g., a needleless syringe) to the needle free connector 154. If needed, the needle free connector 154 may include a priming port 158 that allows the user to prime the extension tubing system 150 and/or the catheter system 100 during use. Additionally or alternatively, the priming port may be used to withdraw fluid from the patient and/or introduce fluid into the patient.

To introduce the catheter 130 into the patient's vasculature, the user may utilize an introducer 160. The introducer 160 may connect (e.g., the system 100 may come with the introducer 160 already connected) to a proximal port 116 on the hub housing 110 and a needle 162 of the introducer 160 may extend through the proximal port 116, the fluid path 111 of the hub housing 110, the flow path 122 of the catheter hub 120 and the lumen 132 of the catheter 130. To secure the introducer 160 to the catheter system 100, the system 100 (e.g., the hub housing 110) may have one or more retention elements 105 extending from a surface of the catheter system 100 (e.g., a surface of the hub housing 110). This retention element(s) may engage with an internal surface of the base 164 of the introducer 160. For example, the internal surface of the base 164 may have a indented area that the retention element 105 snaps into when the introducer 160 is connected to the catheter system 100.

Once the catheter 130 has been inserted into the vasculature, the introducer 160 may be disconnected from the catheter system 100 (e.g., from the hub housing 110). To that end, the base 164 (which has the needle 162 affixed to it) is withdrawn first, leaving a needle cover 166 (e.g., the front piece) in place for the tip of the needle 162 to retract within. At this point, the needle cover 166 releases (e.g., the retention element 105 snaps out the indented area within the introducer 160) and the needle 162 can be disposed safely. An internal mechanism within the needle cover 166 (not shown) is triggered as the needle tip enters the needle cover 166 to lock the needle 162 therein and release the needle cover 166 for disposal of the introducer 160.

In some embodiments, the hub housing 110 may have a seal member 170 located within the proximal port 116 to seal the proximal port, for example, when the introducer 160 is not connected/has been disconnected from the hub housing 110. When the introducer 160 is connected, the needle 162 may pierce the seal member 170 to access the interior of the hub housing 110 and extend through the proximal port 116, the fluid path 111 of the hub housing 110, the flow path 122 of the catheter hub 120 and the lumen 132 of the catheter 130. To prevent leakage after removal of the introducer 160, the seal member 170 may be made from a resilient and self-sealing material that seals any hole created by the needle 162. It should be noted that, although the seal member 170 is shown as a member with an internal cavity 172, any configuration of a seal member 170 may be used to seal the proximal port 116. Also, once the introducer 160 has been removed, the seal member 170 may allow additional medical implements or medical articles to be connected to the proximal port 116 to access the interior of the hub housing 110 and extend into or through the proximal port 116, the fluid path 111 of the hub housing 110, the flow path 122 of the catheter hub 120, the lumen 132 of the catheter 130 and/or the vasculature of the patient.

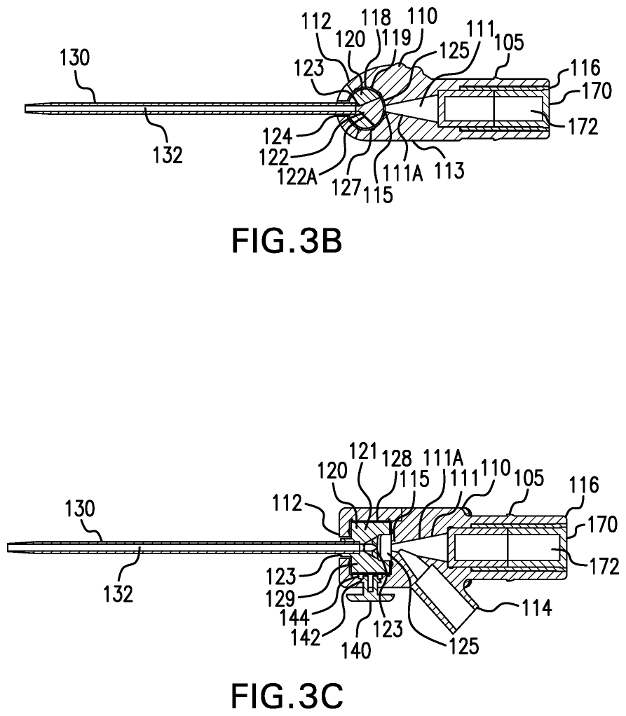

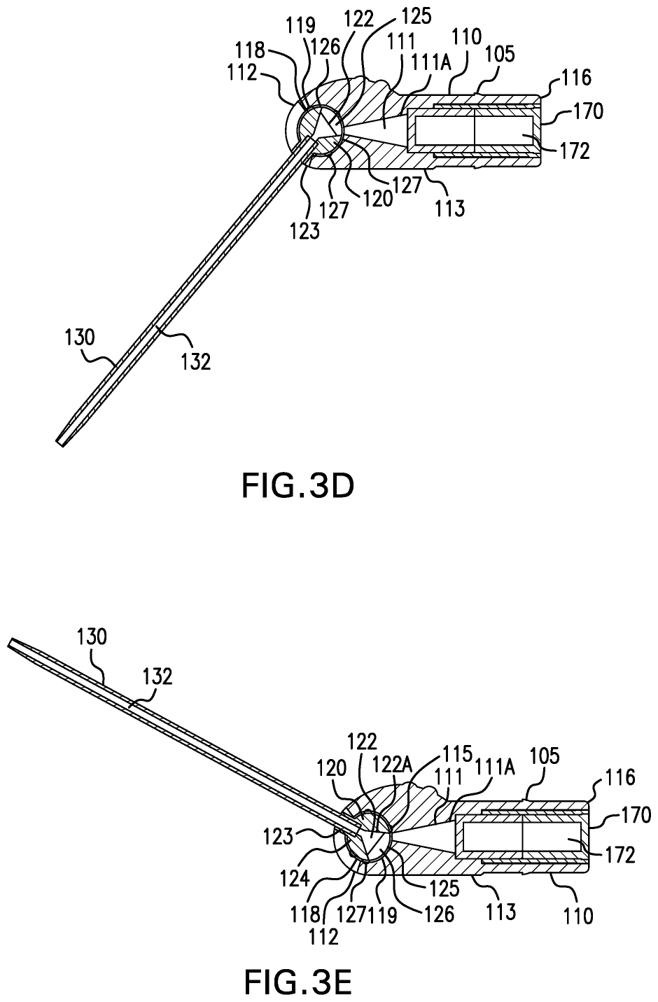

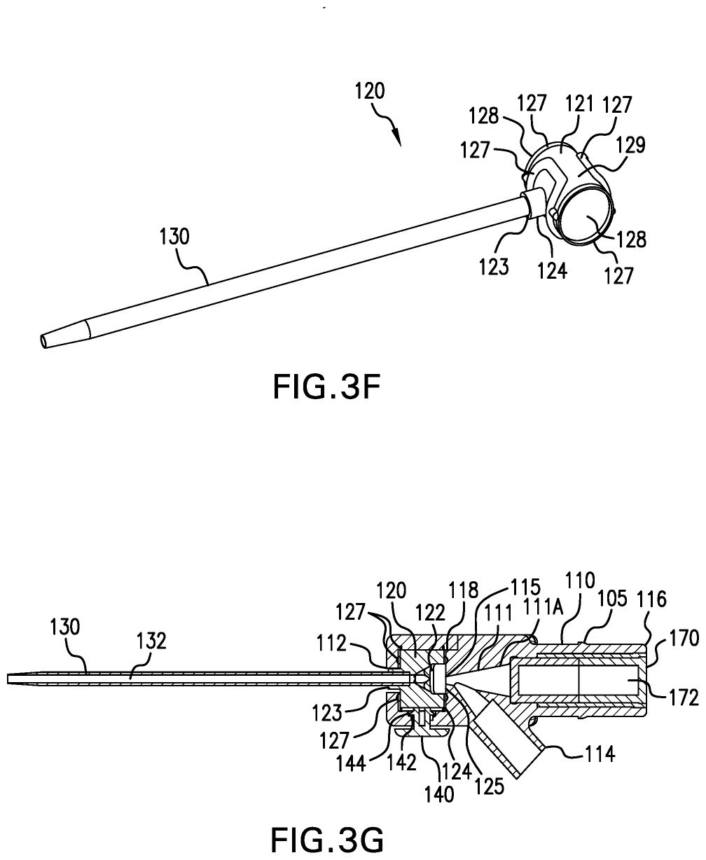

To better illustrate the features and functionality of the catheter system 100, FIGS. 3A to 3G show the catheter system 100 and the components of the catheter system 100 with the introducer 160 removed. As best shown in FIGS. 3B and 3C, the catheter hub 120 may be located within a cavity 118 of the hub housing 110 and wall 119 of the cavity 118 may moveably retain the catheter hub 120 within the hub housing 110. To allow the catheter 130 to extend out of the hub housing 110 (e.g., so that it may be inserted into the patient's vasculature), the hub housing 110 may have an opening 112 (e.g., located distal to the catheter hub 120) through which the catheter 130 may extend. As discussed in greater detail below, the opening 112 may be sized to allow the catheter hub 120 and the catheter 130 to rotate.

As noted above, the catheter hub 120 has an internal flow path 122 that extends through it and the hub housing 110 has a fluid path 111 extending through it to allow fluid to be transferred through the catheter system 100. To that end, the flow path 122 of the catheter hub 120 may extend from an opening 123 at the distal end 124 of the catheter hub 120 to an inlet 125 at the proximal end 126. The lumen 132 of the catheter 130 may be in fluid communication with the opening 123 at the distal end 124 of the catheter hub 120. Similarly, the internal fluid path 111 of the hub housing 110 may extend through a portion of the hub housing 111, and the cavity 118 may be located distal to the fluid path 111. The fluid path 111 has a distal opening 115 that is fluidly connected to the inlet 125 of the flow path 122 in the catheter hub 120 (e.g., when the catheter hub 120 is retained within the cavity 118). The opening 123 at the distal end 124 of the catheter hub 120 may be located proximate the opening 112 of the cavity wall 119. To help guide a medical article (e.g., the needle 162 of the introducer 160 or other medical article attached to the proximal port 116 after removal of the introducer 160) through the catheter system 100 (e.g., through the flow path 122 and the fluid path 111), the flow path 122 and the fluid path 111 may have tapered areas 122A/111A that guide the medical article through the catheter system 100.

During rotation/articulation of the catheter hub 120 within the hub housing 110, for example, between a first position (e.g., FIG. 3B) and a second position (e.g., FIG. 3D and/or 3E), an external surface of the catheter hub 120 (e.g., a member extending from the exterior surface or the exterior surface itself) and the wall 119 (e.g., the interior of the wall) of the cavity 118 movably interact with one another and maintain a fluid tight seal between them to prevent leakage through the catheter system 100. This fluid tight seal may be maintained regardless of the rotational position of the catheter hub 120. For example, as shown in FIG. 3F, the catheter hub 120 (e.g., the exterior surface 129) may have one or more interacting surfaces 127 that extend from the body 121 of the catheter hub 120 and seal against the cavity wall 119 to provide a high pressure seal (e.g., up to 325 PSI or greater) between the fluid path 111, flow path 122, the catheter hub 120 and the hub housing 110 and shield the interior of the hub housing 110 from the environment. These interacting surface(s) 127 may be a resilient material that is overmolded onto the catheter hub 120. Alternatively, the interacting surface(s) 127 may be integral to/unitary with the catheter hub 120. To help with rotation/articulation, in some embodiments, a silicone lubricant may be applied at interacting surface(s) 127.

In some embodiments, the interacting surface(s) may extend from various surfaces of the main body 121 of the catheter hub 120. For example, the interacting surface(s) may extend out from around the opening 123 at the distal end 124 of the flow path 122 and around the inlet 125 at the proximal end 126. Additionally or alternatively, the interacting surface(s) 127 may extend from one or both of the side walls 128 of the catheter hub 120 so that they seal against the sides of the cavity 118.

Various embodiments of the present invention can rotate between any number of positions. For example, when the catheter hub 120 is in a first position, the longitudinal axis of the distal opening 115 of the fluid path 111 in the hub housing 110 may be parallel with the longitudinal axis of the catheter lumen 132 (see FIG. 3B). Conversely when the catheter hub 120 is in the second position, the longitudinal axis of the distal opening 115 of the fluid path 111 in the hub housing 110 may form an angle with the longitudinal axis of the catheter lumen 132 (e.g., and when the bottom surface 113 of the hub housing 110 is placed upon the patient's body) (see FIGS. 3D and 3E). It should be noted that, although the figures show the fluid path 111 in the hub housing 110 in fluid communication with the flow path 122 in the catheter hub 120 in all positions (e.g., FIGS. 3B, 3D and 3E), other embodiments may rotate to positions in which fluid cannot flow through the catheter system 100 (e.g., the fluid path 111 in the hub housing 110 is fluidly disconnected from the flow path 122 in the catheter hub 120). It should be noted that the catheter 130 may rotate through a wide range of angles, for example, from between 20 degrees (e.g., as shown in FIG. 3E) and −45 to −60 degrees (FIG. 3D). Furthermore, it should also be noted that the catheter 130 may articulate in additional plane(s) other than the plane containing the longitudinal axis of the distal opening 115 of the fluid path 111 in the hub housing 110, for instance, by utilizing a ball and socket configuration (not shown).

In some instances it may be necessary and/or desirable to lock the catheter hub 120 in a certain position so that it is not free to rotate/articulate. To that end, some embodiments may have a retaining member 140 that locks the catheter hub 120 in place. For example, as best shown in FIG. 3C and FIG. 3G, the retaining member 140 may extend at least partially through the hub housing 110 and transition between an unlocked position (FIG. 3C) and a locked position (FIG. 3G). When in the unlocked position, the retaining member 140 does not forcibly contact the catheter hub 120 and the catheter hub 120 is free to rotate. When in the locked position, the retaining member 140 may press against the catheter hub 120 and bias the catheter hub 120 against the cavity wall 119. Cavity wall 119 and/or catheter hub 120 may be further configured to help resist relative rotation when in the locked position (e.g. may have texturing, castellations, etc.). If the catheter hub 120 has interacting surface(s) extending from the sides 128 of the catheter hub 120, this biasing action may depress the interacting surface(s) 127 contacting the cavity wall 119. The pressure/resistance created between the catheter hub 120 and the cavity wall 119 will hinder the catheter hub 120 from rotating. Alternatively, the end of the retaining member 140 may enter a recess within the catheter hub 120 to resist rotation. For example, the end of the retaining member may be square (or similar shape that would prevent rotation when keyed in the catheter hub) and may enter a square recess within the catheter hub. To maintain the retaining member 140 within the locked position, the retaining member may have one or more tabs 142 extending from the body of the retaining member. Conversely, the hub housing 110 may have a ridge/recess 144 into which the tab(s) 142 may snap as the retaining member 140 moves from the unlocked position to the locked position.

During use, the user may first insert the catheter 130 into the patient's vasculature using the introducer 160 and then disconnect the introducer 160 as discussed above. Once the catheter 130 is properly located within the vasculature, the user may place the hub housing 110 on the patient, such that the bottom surface 113 is located on the skin of the patient, while also rotating/articulating that catheter system 100 (e.g., the user may rotate the catheter hub 120 within the hub housing 110) such the catheter 130 is extending out of the hub housing 110 at an angle (e.g., such that the longitudinal axis of the distal opening 115 of the fluid path 111 in the hub housing 110 forms an angle with the longitudinal axis of the catheter lumen 132). By rotating the catheter hub 120 in this manner, it allows the catheter 130 to extend out of the hub housing 110 and enter the patient at a more natural angle, reduces the stresses on the catheter 130 at the insertion site, allows the catheter system 100 to conform to the patient's body, and prevents kinking of the catheter 130 at the insertion site. Once the catheter hub 110 and catheter 130 are rotated to the natural angle/position and if the catheter system 100 includes a retaining member 140, the user may then depress the retaining member 140 to lock the catheter hub 120 in the angled position.

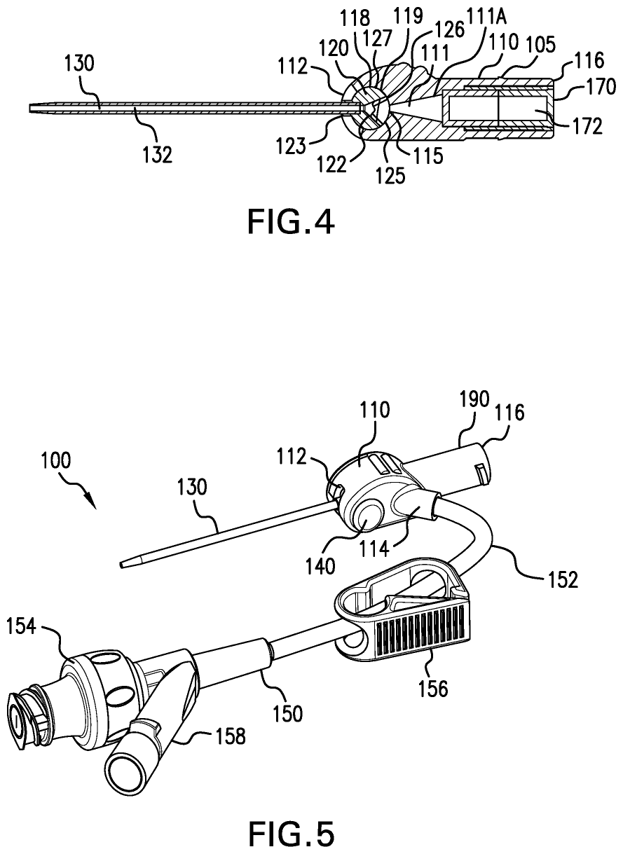

Although the interacting surfaces 127 are described above as extending from the surface of the catheter hub 120, other embodiments may utilize different configurations for the interacting surface. For example, as shown in FIG. 4, the outer/exterior surface 129 of the catheter hub 120 may act as the interacting surface. In such embodiments, the exterior surface 129 of the catheter hub 120 may be aligned with the wall 119 of the cavity 118 within the hub housing 110 such that the exterior surface 129 contacts the wall 119 directly. To help maintain the seal between the hub housing 110 and the catheter hub 120, the catheter hub 120 may be made from a dissimilar material such as a resilient material and the hub housing from a semi-rigid or rigid material.

As discussed above, the hub housing 110 may have a proximal port 116 to which the introducer 160 and to which additional medical articles and medical implements may be connected after disconnection of the introducer. Any number of connections may be used on the proximal port 116 to facilitate connection of the medical article/implement. For example, as shown in FIG. 5, the proximal port 116 may have a standard Luer interface to allow medical articles/implements having Luer connections to easily connect to the catheter system 100.

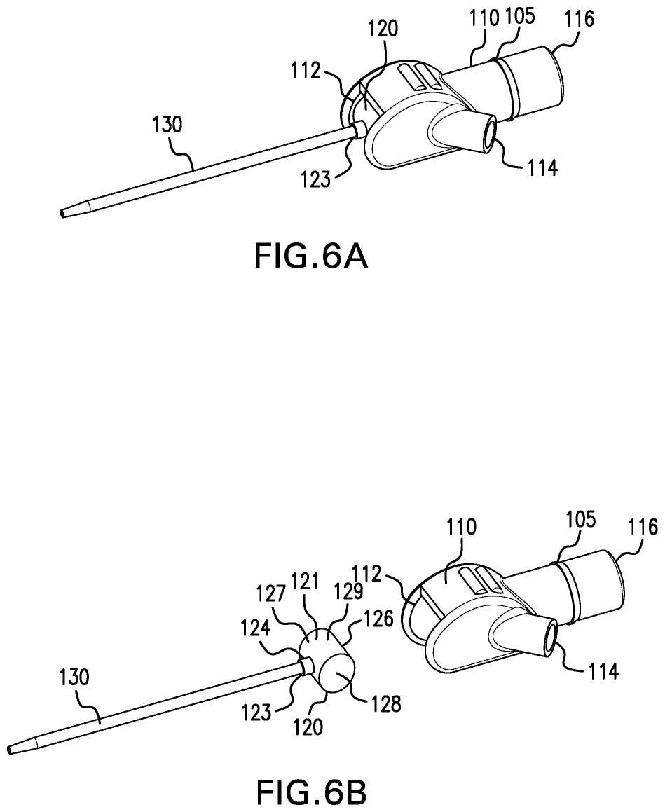

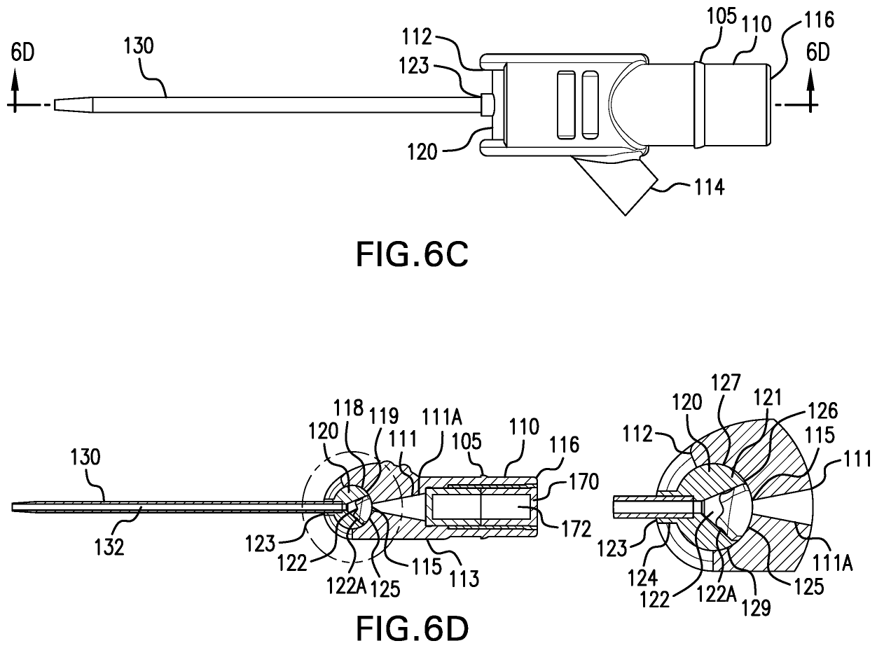

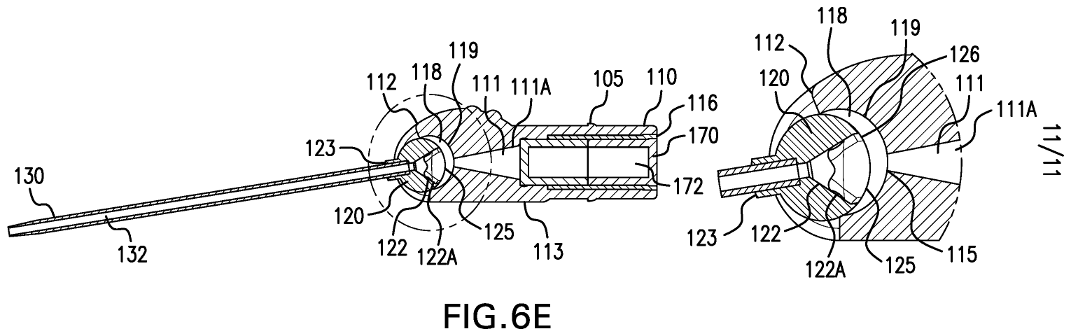

In some instances/applications, it may be beneficial to separate the hub housing 110 from the catheter hub 120, such as to replace the hub housing 110. To that end, FIGS. 6A to 6E show an alternative embodiment of a catheter system 100 that allows the catheter hub 120 to be separated from the hub housing 110. To facilitate this connection and disconnection of the catheter hub 120, the opening 112 in the wall 119 of the hub housing 110 may be enlarged to allow the catheter hub 120 to slide or snap into the cavity 118 of the hub housing 110. The opening 112 must be large enough to allow the catheter hub 120 to enter the cavity 118 without requiring too much force, but small enough to still retain the catheter hub 120 within the cavity. To that end, the opening 112 should be less than 180° in order retain the catheter hub 120 after joining to the hub housing, but remain near 180° to limit the amount of connection force required to join the components. For example, as shown in FIG. 6D, the opening 112 may be approximately 155 degrees. Additionally or alternatively, the opening 112 in the wall 119 of the hub housing 110 may be configured to easily receive the catheter hub 120 when inserted into the cavity 118 in a first orientation but resists releasing the catheter hub 120 from the cavity 118 when in a second orientation inside the cavity 118 (e.g. offset geometries).

The functionality of the embodiment shown in FIGS. 6A to 6E is identical to the embodiments described above. In particular, the user may first insert the catheter 130 into the patient's vasculature using the introducer 160 and then disconnect the introducer 160 as discussed above. The user may then place the hub housing 110 on the patient such the bottom surface 113 is located on the skin of the patient while rotating/articulating that catheter system 100 (e.g., the user may rotate the catheter hub 120 within the hub housing 110) such the catheter 130 is extending out of the hub housing 110 at an angle (e.g., such that the longitudinal axis of the distal opening 115 of the fluid path 111 in the hub housing 110 forms an angle with the longitudinal axis of the catheter lumen 132) (FIG. 6E, which shows the catheter hub 120 being connected/partially connected to the hub housing 110). Once the catheter hub 110 and catheter 130 are rotated to the intended angle/position and if the catheter system 100 includes a retaining member 140, the user may then depress the retaining member 140 to lock the catheter hub 120 in the correct position.

It should be noted that FIGS. 6A to 6E show the catheter hub 120 configuration shown within FIG. 4 (e.g., with the exterior surface 129 of the catheter hub 120 aligned with the wall 119 of the cavity 118 within the hub housing 110). However, alternative embodiments may use the catheter hub 120 shown in FIG. 3F and having the overmolded/two-shot interacting surfaces 127.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A catheter system for conforming to a patient's body following insertion therein, the catheter system comprising:
a catheter hub having a proximal end, a distal end, an exterior surface, and an internal flow path extending from an opening at the distal end to an inlet at the proximal end, the flow path configured to allow fluid to flow between the distal end and the proximal end of the catheter hub;
a catheter shaft having a lumen with a longitudinal axis, the lumen in fluid communication with the opening at the distal end of the catheter hub; and
a hub housing having a bottom surface, an internal fluid path extending through at least a portion of the hub housing, and a cavity located distal to the fluid path and having a wall with an opening, the wall of the cavity configured to movably retain the catheter hub, the fluid path having a distal opening with a longitudinal axis and being fluidly connected to the inlet of the flow path in the catheter hub when the catheter hub is retained within the cavity of the hub housing, the opening at the distal end of the catheter hub located proximate the opening of the cavity wall,
wherein the external surface of the catheter hub and the wall of the cavity are configured to movably interact and maintain a fluid tight seal therebetween from a first position to at least a second position, the longitudinal axis of the fluid path distal opening being parallel with the longitudinal axis of the catheter shaft lumen when in the first position, the longitudinal axis of the fluid path distal opening forming an angle with the longitudinal axis of the catheter shaft lumen when in the second position and when the bottom surface of the hub housing is placed upon the patient's body.

2. A catheter system according to claim 1, further comprising:
a retaining member extending at least partially through the hub housing and configured to transition between a locked position and an unlocked position, the retaining member configured to selectively lock the catheter hub in the first or second position when in the locked position.

3. A catheter system according to claim 2, wherein the retaining member acts on the catheter hub when in the locked position to press the catheter hub against a retaining surface, thereby locking the catheter hub.

4. A catheter system according to claim 3, wherein the retaining surface is an internal surface within the hub housing.

5. A catheter system according to claim 1, wherein the hub housing further includes:
a proximal port located at a proximal end of the hub housing.

6. A catheter system according to claim 5, wherein the proximal port includes a seal member located within the proximal port, the seal member sealing the proximal port.

7. A catheter system according to claim 5, wherein the proximal port includes a luer connector.

8. A catheter system according to claim 5, further comprising:
an introducer configured to connect to the proximal port of the hub housing, the introducer having a needle extending through the internal fluid path, the internal flow path, and the lumen of the catheter shaft when the introducer is connected to the proximal port, the introducer configured to allow the catheter to be inserted into a patient's vasculature when connected and configured to be disconnected from the proximal port after insertion of the catheter into the patient's vasculature.

9. A catheter system according to claim 8, wherein the hub housing includes at least one retention element extending from an exterior surface of the hub housing, the at least one retention element configured to engage the introducer to secure the introducer to the hub housing when connected.

10. A catheter system according to claim 1, wherein the external surface of the catheter hub includes at least one interacting surface extending from the external surface, the at least one interacting surface configured to contact the wall of the cavity to maintain the fluid tight seal.

11. A catheter system according to claim 10, wherein the at least one interacting surface is a resilient material overmolded on the catheter hub.

12. A catheter system according to claim 1, wherein the exterior surface of the catheter hub is aligned with the wall of the cavity, thereby maintaining the fluid tight seal.

13. A catheter system according to claim 1, wherein the hub housing includes a side port, the side port fluidly connected to an extension tubing system.

14. A catheter system according to claim 13, wherein the extension tubing system includes:
a needle free connector having an inlet, an outlet and a priming port; and
a tube having a first end fluidly connected to the outlet of the needle free connector and a second end connected to the side port of the hub housing.

15. A catheter system according to claim 1, wherein the opening is shaped to receive the catheter hub such that the catheter hub may be connected and/or disconnected from the hub housing.

16. A method for connecting a catheter to a patient's vasculature comprising:
providing a catheter system, the catheter system including:
a catheter hub having a proximal end, a distal end, an exterior surface, and an internal flow path extending from an opening at the distal end to an inlet at the proximal end, the flow path configured to allow fluid to flow between the distal end and the proximal end of the catheter hub,
a catheter shaft having a lumen with a longitudinal axis, the lumen in fluid communication with the opening at the distal end of the catheter hub, and
a hub housing having a bottom surface, an internal fluid path extending through at least a portion of the hub housing, and a cavity located distal to the fluid path and having a wall with an opening, the wall of the cavity configured to movably retain the catheter hub, the fluid path having a distal opening with a longitudinal axis and being fluidly connected to the inlet of the flow path in the catheter hub when the catheter hub is retained within the cavity of the hub housing, the opening at the distal end of the catheter hub located proximate the opening of the cavity wall;
inserting the catheter shaft into the patient's vasculature; and
rotating the catheter hub with respect to the hub housing from a first position to a second position, the longitudinal axis of the fluid path distal opening being parallel with the longitudinal axis of the catheter shaft lumen when in the first position, the longitudinal axis of the fluid path distal opening forming an angle with the longitudinal axis of the catheter shaft lumen when in the second position and when the bottom surface of the hub housing is placed upon the patient's body.

17. A method according to claim 16, wherein the external surface of the catheter hub and the wall of the cavity movably interact and maintain a fluid tight seal therebetween during rotation from the first position to the at least a second position.

18. A method according to claim 16, wherein hub housing includes a proximal port located at a proximal end of the hub housing.

19. A method according to claim 18, wherein inserting the catheter shaft into the patient's vasculature includes:
using an introducer to insert the catheter shaft into the patient's vasculature, the introducer connected to the proximal port of the hub housing and having a needle extending through the internal fluid path, the internal flow path, and the lumen of the catheter shaft when the introducer is connected to the proximal port;
inserting the needle and catheter shaft into the patient's vasculature; and
disconnecting the introducer from the proximal port of the hub housing after the catheter is inserted into the patient's vasculature.

20. A method according to claim 16, further comprising:
transitioning a retaining member from an unlocked position to a locked position, the retaining member extending at least partially through the hub housing and locking the catheter hub in the second position when in the locked position.
